(19) United States
(12) Patent Application Publication
(10) Pub. No.: US 2013/0105785 A1
(43) Pub. Date: May 2, 2013

(54) NOVEL ORGANIC COMPOUND AND ORGANIC LIGHT-EMITTING DEVICE INCLUDING THE SAME

(71) Applicants: Masanori Seki, Yokohama-shi (JP); Ryuji Ishii, Yokohama-shi (JP); Hajime Muta, Zama-shi (JP)

(72) Inventors: Masanori Seki, Yokohama-shi (JP); Ryuji Ishii, Yokohama-shi (JP); Hajime Muta, Zama-shi (JP)

(73) Assignee: CANON KABUSHIKI KAISHA, Tokyo (JP)

(21) Appl. No.: 13/807,836

(22) PCT Filed: Jul. 1, 2011
(86) PCT No.: PCT/JP2011/065649

§ 371 Date: Dec. 31, 2012

(30) Foreign Application Priority Data
Jul. 6, 2010 (JP) 2010-153989

(51) Int. Cl. H01L 51/50; C07D 487/04

(52) U.S. Cl. CPC H01L 51/5012; C07D 487/04
USPC 257/40; 548/433

(57) ABSTRACT

The present invention provides a blue or green phosphorescent organic electroluminescent device which has high luminous efficiency. An organic electroluminescent device includes a light-emitting layer containing a pyrroloindole compound represented by general formula (1) below.

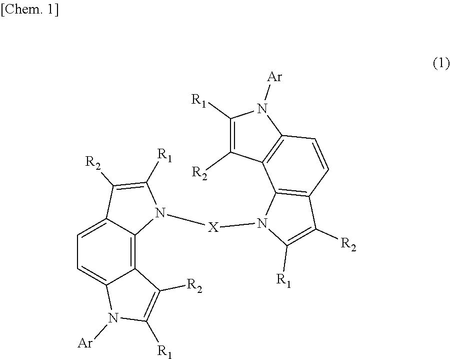

In general formula (1), X represents a substituted or unsubstituted arylene group, Ar₁ and Ar₂ each represent a substituted or unsubstituted aryl group, and R₁ to R₈ each represent a hydrogen atom or an alkyl group having 1 to 2 carbon atoms.

### TECHNICAL FIELD

The present invention relates to a pyrroloindole compound, which is a novel compound, and also relates to an organic light-emitting device including the novel compound.

### BACKGROUND ART

An organic light-emitting device has a structure in which a pair of opposing upper and lower electrodes are disposed on a transparent substrate and organic compound layers including a light-emitting layer are stacked between the electrodes. Organic light-emitting devices have been receiving attention as a technology to realize next-generation full-color displays having high-speed responsiveness, high luminous efficiency, and flexibility, and material and device technologies thereof have been actively under development. Among the organic light-emitting devices, in particular, those which utilize electroluminescence may be referred to in some cases as organic electroluminescent devices, organic EL devices, or organic electroluminescence devices.

In recent years, in order to enhance luminous efficiency of devices, organic light-emitting devices utilizing phosphorescence via triplet excitons (hereinafter, referred to as “phosphorescent devices”) have been actively under development. As the light-emitting material, from the standpoint of material stability and luminous efficiency, a metal complex containing iridium (Ir), such as FIrPic (bis(3,5-difluoro-2-(2-pyridyl)phenyl-(2-carboxypyridyl)iridium III), is used.

When an iridium complex is used as a light-emitting material (guest material), it is important to select a suitable host material for the guest material. It is required that the lowest excited triplet level (T₁) of the host material be higher than the T₁ of the guest material.

Devices with a higher-luminance light output or high conversion efficiency are required under the present circumstances.

In addition, there are still many problems to be solved in terms of durability, such as changes with time when used for a long period of time and degradation due to an atmospheric gas including oxygen, humidity, or the like.

Furthermore, in the case of application to full-color displays and the like, light emission of blue, green, and red with good color purity is required. However, these problems have not yet been solved satisfactorily.

PTL 1 discloses an organic electroluminescent device in which an indolocarbazole compound is used as a hole-transporting material. The indolocarbazole compound has a hole-transporting capability derived from carbazole which is a partial skeleton. However, since its electron-transporting capability is not large, use of the indolocarbazole compound is limited to the layer that is responsible for hole injection or transport. Furthermore, because of its low T₁ value, the indolocarbazole compound is inadequate as a host material for the blue light-emitting layer of a phosphorescent device. A hole-transporting host material having a higher T₁ value has been desired.

### CITATION LIST

### Patent Literature

PTL 1 U.S. Pat. No. 5,942,340

### Non Patent Literature

NPL 1 Eur. J. Med. Chem. 37, 261-266 (2002)

### SUMMARY OF INVENTION

### Technical Problem

The present invention provides a novel organic compound. The present invention also provides an organic light-emitting device which has high luminous efficiency and which is capable of low-voltage driving.

### Solution to Problem

A novel organic compound according to the present invention is a pyrroloindole compound represented by general formula (1) below.

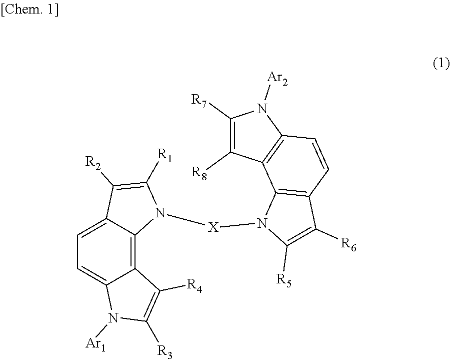

In general formula (1), X represents a substituted or unsubstituted arylene group, Ar₁ and Ar₂ each represent a substituted or unsubstituted aryl group, and R₁ to R₈ each represent a hydrogen atom or an alkyl group having 1 to 2 carbon atoms.

An organic light-emitting device according to the present invention includes at least one organic layer disposed between a pair of opposing electrodes, in which at least one of the at least one organic layer is a light-emitting layer containing the pyrroloindole compound represented by general formula (1) above.

### Advantageous Effects of Invention

According to the present invention, it is possible to provide a novel compound which is useful as a host material for a phosphorescent device. It is also possible to provide an organic light-emitting device which has high luminous efficiency and which can be driven at low voltage.

### BRIEF DESCRIPTION OF DRAWINGS

FIG. 1 is a schematic cross-sectional view showing organic light-emitting devices and switching devices connected to the organic light-emitting devices.

### DESCRIPTION OF EMBODIMENTS

A novel organic compound according to the present invention is a pyrroloindole compound represented by general formula (1) below.

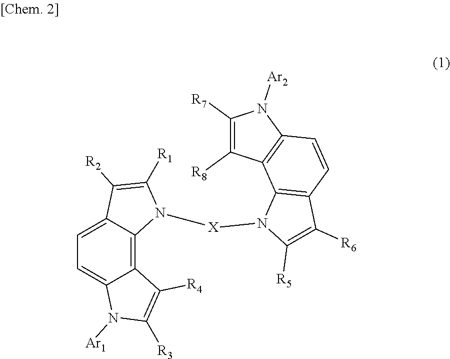

In general formula (1), X represents a substituted or unsubstituted arylene group, Ar₁ and Ar₂ each represent a substituted or unsubstituted aryl group, and R₁ to R₈ each represent a hydrogen atom or an alkyl group having 1 to 2 carbon atoms.

As a result of diligent research in order to solve the above-mentioned problems, the present inventors have found a pyrroloindole compound of the present invention. Furthermore, by using the pyrroloindole compound of the present invention as a host material for a phosphorescent device, there is provided an organic light-emitting device which has high luminous efficiency and which can be driven at low voltage.

The reason for the fact that the organic electroluminescent device using the pyrroloindole compound according to the present invention has high luminous efficiency originates in the following characteristics of the pyrroloindole compound:

[1] The triplet energy level (T₁) is high at 450 nm or less. Therefore, the pyrroloindole compound can be used as a host material in the case where a phosphorescent Ir metal complex that emits green light (emission peak: 480 to 530 nm) or a phosphorescent Ir metal complex that emits blue light (emission peak: 450 to 470 nm) is used as a guest material. Note that the triplet energy level (T₁) is defined as the phosphorescence 0-0 band at the temperature of 77 K in a toluene solution.

[2] The highest occupied molecular orbital (HOMO) energy level (hereinafter, abbreviated as “HOMO level”) is high. The HOMO level of the pyrroloindole compound of the present invention is higher than −5.7 eV. A material having a HOMO energy level higher than −5.7 eV is used for an adjacent layer (e.g. a hole transport layer composed of a hole-transporting material) adjacent to the light-emitting layer. Consequently, when used as a host material, the HOMO level of the host material desirably has a HOMO level higher than −5.7 eV so that hole injection is efficiently performed from the adjacent layer to the light-emitting layer. Furthermore, because of the high HOMO level, the pyrroloindole compound can also be used as a hole injection and transport material.

The pyrrole group and the indole group in the structure of the compound of the present invention are important for exhibiting the characteristics [1] and [2] described above. The pyrrole group and the indole group have high HOMO levels, and since the compound has these groups in its skeletal structure, the T₁ value is high.

In addition, by defining each of R₁ to R₈ in general formula (1) as a hydrogen atom or an alkyl group having 1 to 2 carbon atoms, the compound enables lower voltage operation and high mobility can be maintained.

In general formula (1), X represents a substituted or unsubstituted arylene group. Examples of the substituted or unsubstituted arylene group include a phenylene group, a biphenylene group, a terphenylene group, and a fluorenylene group.

Ar₁ and Ar₂ each represent a substituted or unsubstituted aryl group, and examples thereof include a phenyl group, a biphenyl group, a fluorenyl group, and a terphenyl group. Examples of the biphenyl group include an o-biphenyl group and an m-biphenyl group. Examples of the fluorenyl group include a 1-fluorenyl group, a 3-fluorenyl group, and a 4-fluorenyl group. Examples of the terphenyl group include o-terphenyl and m-terphenyl. Ar₁ and Ar₂ may be the same or different.

X and the aryl group in each of Ar₁ and Ar₂ may be substituted with a substituent to the extent that maintains the characteristics described above. Examples of the substituent include halogen groups, such as fluorine; alkyl groups, such as a methyl group, an ethyl group, an n-propyl group, an n-butyl group, an n-pentyl group, an n-hexyl group, an iso-propyl group, an iso-butyl group, a sec-butyl group, a tert-butyl group, and a cyclo-hexyl group; and alkoxy groups, such as a methoxy group, an ethoxy group, and a propoxy group.

R₁ to R₈ each independently represent a hydrogen atom or an alkyl group having 1 to 2 carbon atoms. Examples of the alkyl group include a methyl group and an ethyl group. A configuration can be selected in which R₁, R₃, R₅, and R₇ each are a methyl group, and R₂, R₄, R₆, and R₈ each are a hydrogen atom. This configuration exhibits an effect of protecting the a position of nitrogen, which is an active site.

Specific examples of the pyrroloindole compound of the present invention will be shown below. However, it is to be understood that the present invention is not limited thereto.

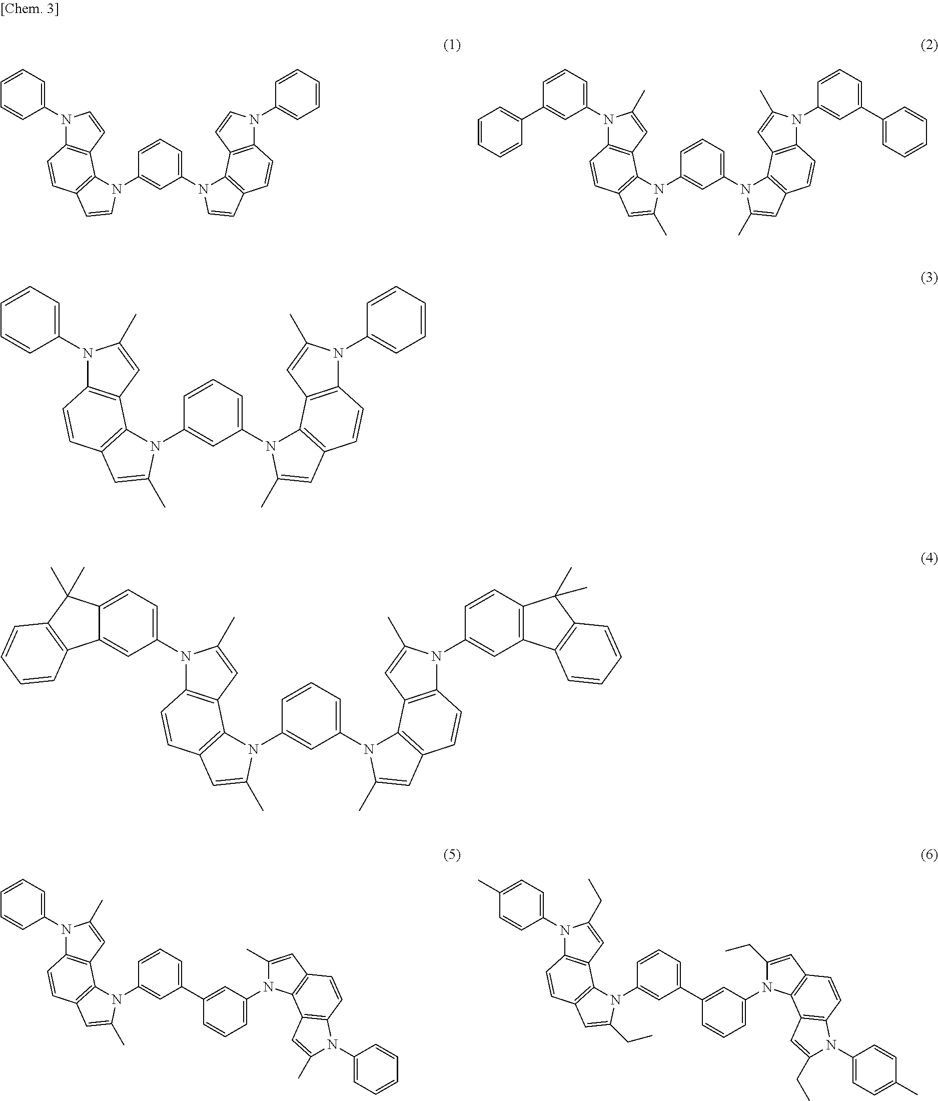

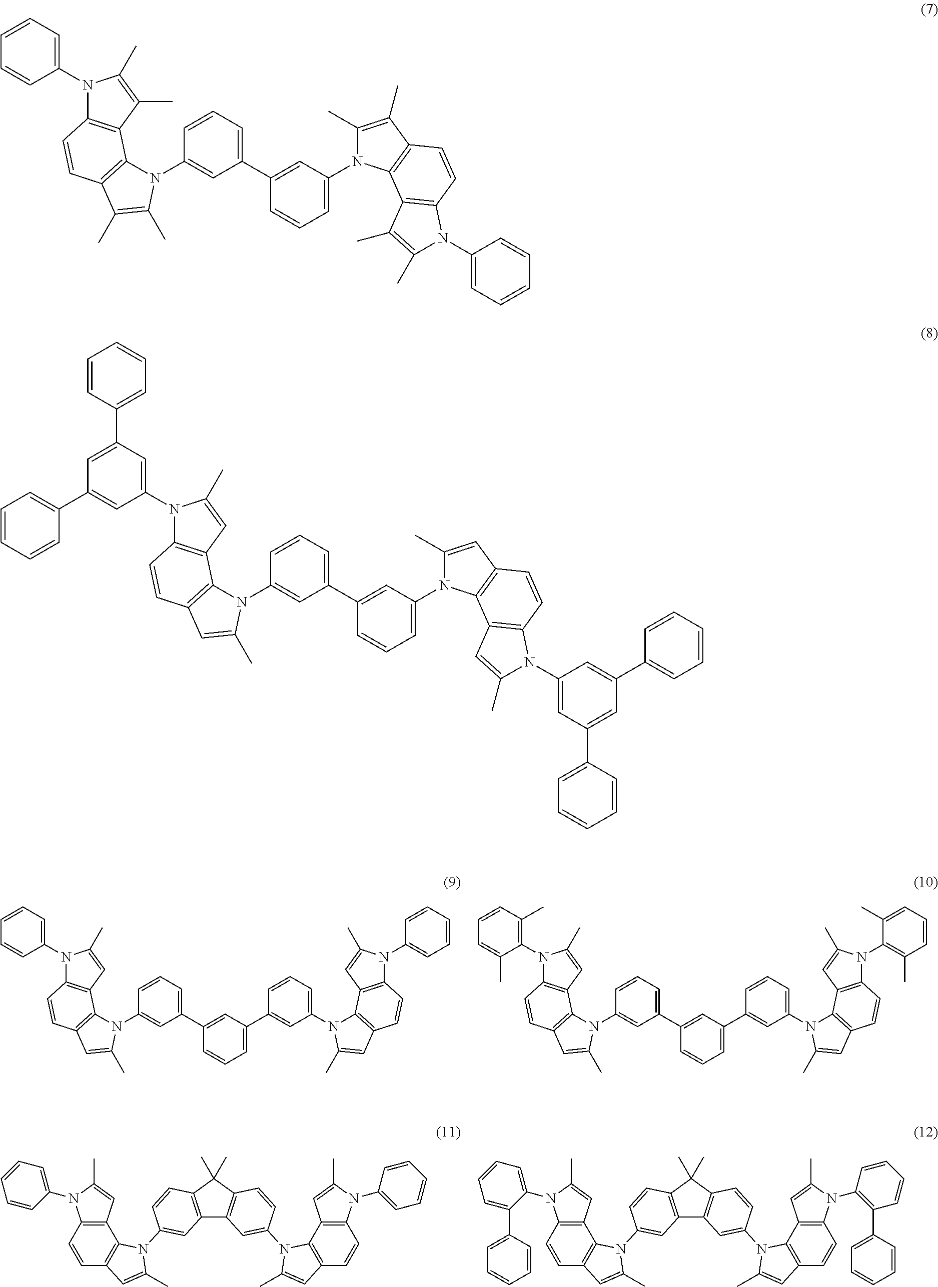

### Synthesis of Organic Compound

The organic compound according to the present invention can be synthesized, for example, by the synthesis route shown below, as described in detail later in Example 1.

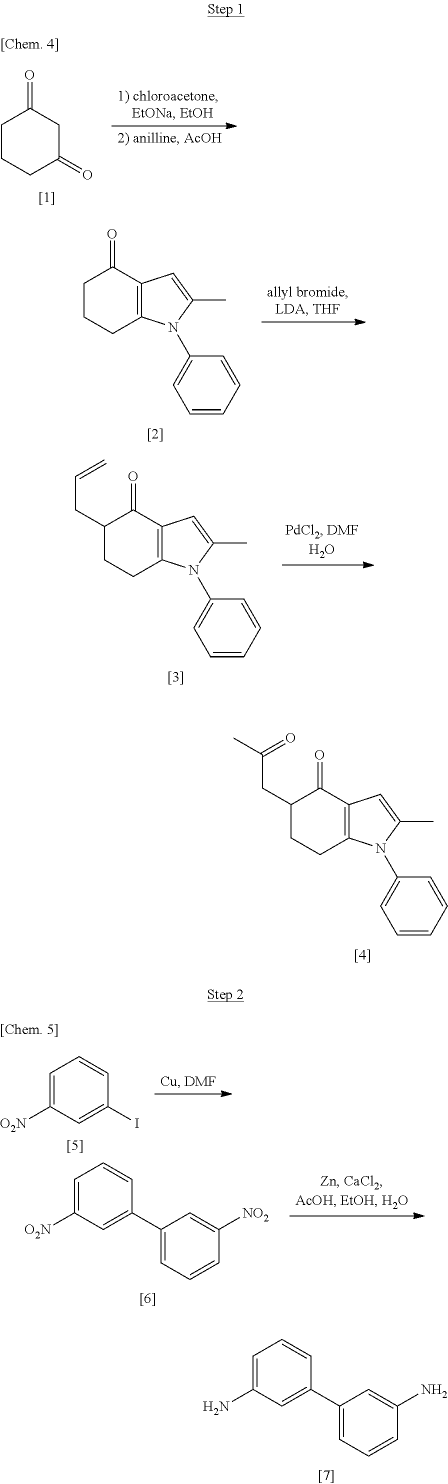

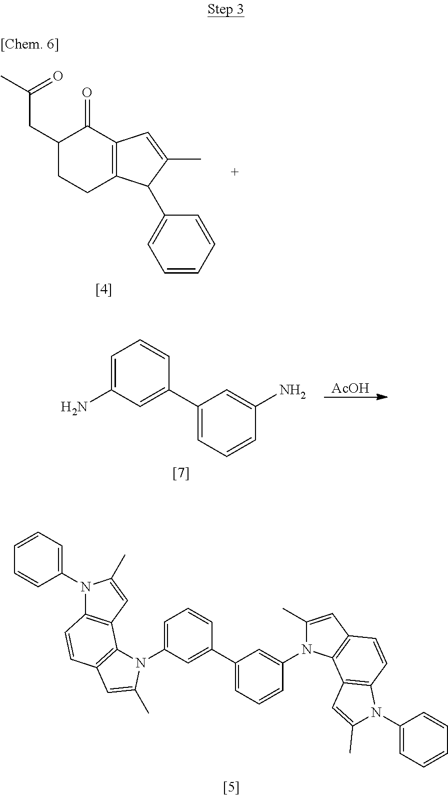

Specifically, in Step 1, following the synthesis method described in NPL 1, an intermediate [4] is synthesized in four steps from a starting material [1] (1,3-cyclohexanedione).

In Step 2, an intermediate [7] is synthesized from a starting material [5]. In Step 3, by reacting the resulting intermediates [4] and [7] with each other, intended exemplary compound (5) can be synthesized.

By changing the starting material [1] in Step 1 and the starting material [5] in Step 2, each of the pyrroloindole compounds of the present invention shown above can be synthesized.

### Description of Organic Light-Emitting Device

An organic light-emitting device according to an embodiment of the present invention will now be described.

An organic light-emitting device according to the embodiment includes at least one organic layer disposed between a pair of opposing electrodes, in which at least one of the at least one organic layer is a light-emitting layer containing a pyrroloindole compound represented by general formula (1) above.

Examples of the structure of an organic light-emitting device according to the present invention include a structure including anode/light-emitting layer/cathode disposed in that order on a substrate; a structure including anode/hole transport layer/electron transport layer/cathode disposed in that order; a structure including anode/hole transport layer/light-emitting layer/electron transport layer/cathode disposed in that order; a structure including anode/hole injection layer/hole transport layer/light-emitting layer/electron transport layer/cathode disposed in that order; and a structure including anode/hole transport layer/light-emitting layer/hole and exciton blocking layer/electron transport layer/cathode disposed in that order. The slash (/) indicates that layers in front and behind the slash are adjacent to each other. However, it is to be understood that these five multilayer structures are merely basic device structures, and the structure of the organic light-emitting device using the compound according to the present invention is not limited thereto. For example, a structure in which an insulating layer is provided at the interface between the electrode and the organic compound layer, a structure in which a bonding layer or interference layer is provided, a structure in which the electron transport layer or hole transport layer includes two layers having different ionization potentials, or other various layer structures may be used.

The light-emitting material (guest material) used in the organic layer of the present invention is not particularly limited as long as it is a material which fluoresces at normal temperature (delayed fluorescent material) or a material which phosphoresces at normal temperature. From the viewpoint of luminous efficiency (external quantum efficiency of the organic light-emitting device) and stability to heat or environment (water and oxygen), an Ir metal complex which phosphoresces at normal temperature can be used.

Specific examples of the phosphorescent Ir metal complex include FIrpic, FIr6, and the Ir metal complex represented by structural formula [Chem. 9] described later.

Besides the light-emitting material, a hole-transporting material and an electron-transporting material are also used. Examples of the hole-transporting material include triarylamine derivatives, phenylenediamine derivatives, triazole derivatives, oxadiazole derivatives, imidazole derivatives, pyrazoline derivatives, pyrazolone derivatives, oxazole derivatives, fluorenone derivatives, hydrazone derivatives, stilbene derivatives, phthalocyanine derivatives, porphyrin derivatives, poly(vinylcarbazole), poly(silylene), and poly(thiophene).

Examples of the electron-transporting material include organic compounds, such as pyridine derivatives, oxadiazole derivatives, oxazole derivatives, triazole derivatives, thiadiazole derivatives, pyrazine derivatives, triazole derivatives, triazine derivatives, perylene derivatives, quinoline derivatives, quinoxaline derivatives, fluorenone derivatives, anthrone derivatives, phenanthroline derivatives, and organic metal complexes, e.g., quinolinol aluminum complexes.

As necessary, the electron-injecting material or electron-transporting material may be used together with a known metal, metal salt, metal oxide, or the like, or a mixture thereof.

Specific examples of the metal, metal salt, or metal oxide include metals, such as lithium, sodium, potassium, cesium, calcium, magnesium, aluminum, indium, silver, lead, tin, and chromium; metal fluorides, such as lithium fluoride and aluminum fluoride; and metal carbonates, such as cesium carbonate.

In the organic light-emitting device of the present invention, a material having a work function that is as large as possible can be used as the material constituting the anode. Examples thereof include elemental metals, such as gold, silver, platinum, nickel, palladium, cobalt, selenium, and vanadium; alloys of these elemental metals; and metal oxides, such as tin oxide, zinc oxide, indium tin oxide (ITO), and indium zinc oxide. Furthermore, conductive polymers, such as polyaniline, polypyrrole, polythiophene, and polyphenylene sulfide, may be used. These electrode materials may be used alone or in combination of two or more. The anode may include a single layer or multiple layers.

A material having a small work function can be used as the material constituting the cathode. Examples thereof include elemental metals, such as lithium, sodium, potassium, cesium, calcium, magnesium, aluminum, indium, silver, lead, tin, and chromium; alloys including two or more of these elemental metals; and salts thereof. Metal oxides, such as indium tin oxide (ITO), can also be used. The cathode may include a single layer or multiple layers.

As the substrate for the organic light-emitting device of the present invention, a non-transparent substrate, such as a metal substrate or a ceramic substrate, or a transparent substrate, such as glass, quartz, or a plastic sheet, is used, although not particularly limited thereto. Furthermore, it is also possible to control luminescent color by providing a color filter film, a fluorescent color conversion filter film, a dielectric reflective film, or the like on the substrate.

The organic light-emitting device of the present invention can be finally covered with a protective layer. As the material for the protective layer, any material that has a function of preventing substances which accelerate degradation of the device, such as moisture and oxygen, from entering the device may be used. Examples of the material constituting the protective layer include, as inorganic materials, nitrides (e.g., SiNₓ and SiₓN_{y}), SiO₂, and Al₂O₃; and, as organic materials, epoxy resins, acrylic resins, urethane resins, polycarbonate, polyether sulfide, and cyclic amorphous polyolefin (COP).

In the protective layer of the organic light-emitting device of the present invention, the inorganic material and the organic material can be used in combination. In the case of combined use, an inorganic protective layer may be formed using the inorganic material, and then an organic protective layer may be formed using the organic material. Alternatively, the organic material and the inorganic material may be mixed to form a protective layer. Basically, the inorganic material blocks the entry of moisture, and the organic material protects the inorganic material and blocks water and oxygen. Thereby, the moisture content inside the device can be maintained at 1 ppm or less.

The method for forming the protective layer covering the organic light-emitting device is not particularly limited. For example, vacuum vapor deposition, sputtering, reactive sputtering, a molecular beam epitaxy (MBE) method, a cluster ion beam method, ion plating, a plasma polymerization method (high-frequency excited ion plating), plasma enhanced CVD, laser assisted CVD, thermal CVD, gas source CVD, a coating method, a printing method, or a transfer method can be used.

In the organic light-emitting device according to the present invention, layers containing the fused polycyclic aromatic compound according to the present invention are generally formed by vacuum vapor deposition or an application method in which the compound is dissolved in an appropriate solvent and applied to form a thin film. Examples of the application method for thin-film formation include a spin coating method, a slit coating method, a printing method, an ink jet method, and a spray method.

In the organic light-emitting device according to the present invention, light extraction efficiency, color purity, and the like can be improved using various known techniques. For example, by processing the surface shape of the substrate (e.g., forming a fine irregular pattern), controlling the refractive indices of the substrate, the ITO layer, and the organic layer, and controlling the thickness of the substrate, the ITO layer, and the organic layer, light extraction efficiency and external quantum efficiency can be improved. Furthermore, by using a microcavity structure (microresonator structure) to reduce unnecessary wavelength components, and by providing a color filter to obtain desired color, the color purity can be improved.

### Use of Organic Light-Emitting Device

The organic light-emitting device according to the present invention can be used for an image display apparatus and an illumination apparatus. Other uses include an exposure light source of an electrophotographic image forming apparatus, a backlight of a liquid crystal display apparatus, and the like.

The image display apparatus includes the organic light-emitting device according to the embodiment provided in a display. The display includes a plurality of pixels. Each pixel includes the organic light-emitting device according to the embodiment and a thin-film transistor (TFT) device, which is an example of a switching device for controlling luminance, and an anode or a cathode of the organic light-emitting device is connected to a drain electrode or a source electrode of the TFT device. The thin-film transistor device serves as a device configured to apply an electrical current to the organic light-emitting device. The display apparatus can be used as an image display apparatus of a PC or the like.

The image display apparatus may be an image output apparatus having an image input portion to which information from an area CCD, a linear CCD, a memory card, or the like is input and configured to output the input image to a display. Furthermore, as a display included in an image pickup apparatus or an ink jet printer, the display apparatus may have both an image output function of displaying an image on the basis of image information input from the outside and an input function of inputting image processing information as an operation panel. Furthermore, the display apparatus may be used as a display of a multifunctional printer.

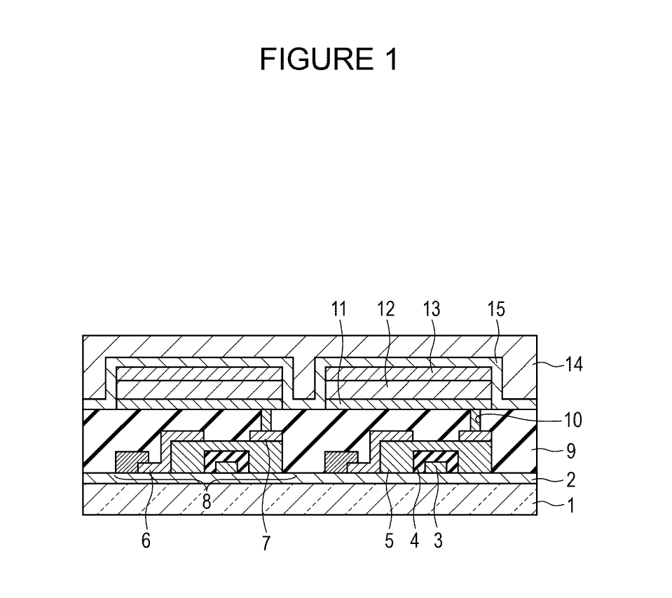

A display apparatus including an organic light-emitting device according to the embodiment will now be described with reference to FIG. 1.

FIG. 1 is a schematic cross-sectional view of an image display apparatus, showing organic light-emitting devices according to the embodiment and thin-film transistor (TFT) devices, as an example of switching devices, which are connected to the organic light-emitting devices. In FIG. 1, an organic light-emitting device and a TFT device constitute one unit, and two units are shown. Details of the structure will be described below.

A display apparatus shown in FIG. 1 includes a substrate 1 composed of glass or the like and a moisture-proof film 2 provided on the substrate 1 in order to protect TFT devices or organic compound layers. Reference numeral 3 denotes a gate electrode composed of a metal. Reference numeral 4 denotes a gate-insulating film, and reference numeral 5 denotes a semiconductor layer.

A TFT device 8 includes the semiconductor layer 5, a drain electrode 6, and a source electrode 7. An insulating film 9 is provided on the TFT device 8. An anode 11 of the organic light-emitting device is connected to the source electrode 7 through a contact hole 10. The structure of the display apparatus is not limited to this as long as one of the anode and the cathode is connected to one of the source electrode and the drain electrode of the TFT device.

In FIG. 1, a multiple-layered organic compound layer 12 is shown as a single layer. A first protective layer 14 and a second protective layer 15 are provided on a cathode 13 in order to suppress degradation of the organic light-emitting device.

In the display apparatus according to the embodiment, the switching device is not particularly limited. A single-crystal silicon substrate, an MIM device, an a-Si type device, or the like may be used.

### EXAMPLES

The present invention will now be described in detail on the basis of examples. It is to be understood that the present invention is not limited thereto.

### Example 1

### Production of Exemplary Compound (5)

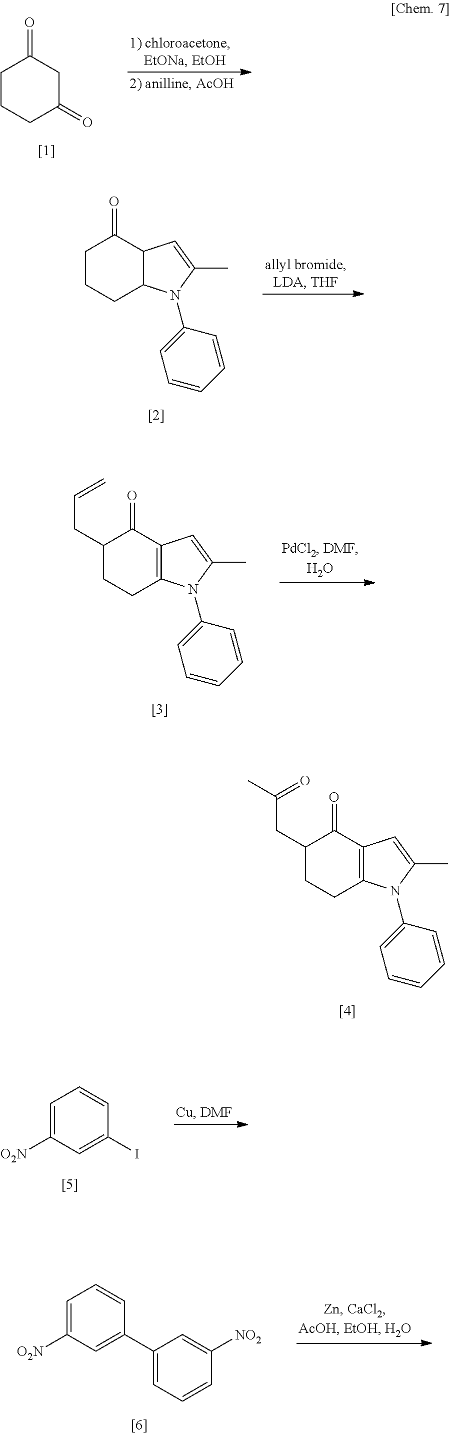 [SMILES: O=C1CCCC(=O)C1 ; CC1=CC2C(=O)CCCC2N1c1ccccc1 ; C=CCC1CCc2c(cc(C)n2-c2ccccc2)C1=O ; CC(=O)CC1CCc2c(cc(C)n2-c2ccccc2)C1=O ; O=[N+]([O-])c1cccc(I)c1 ; O=[N+]([O-])c1cccc(-c2cccc([N+](=O)[O-])c2)c1]

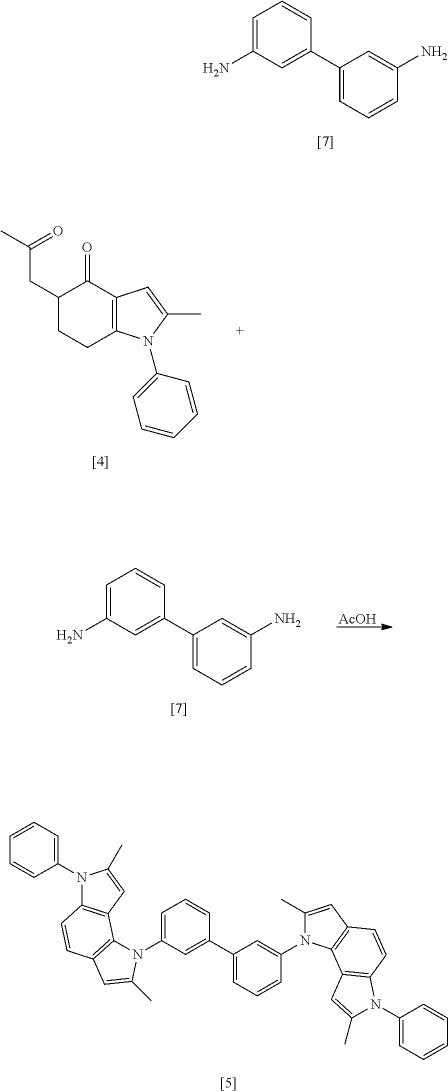

### Synthesis of Intermediate [2]

Following the synthesis method described in NPL 1, 9.00 ml (112 mmol) of chloroacetone was added dropwise to an ethanol (120 ml) solution including 5.0 g (102 mmol) of 1,3-cyclohexanedione [1] (manufactured by Tokyo Chemical Industry Co., Ltd.) and 7.15 g (133 mmol) of sodium ethoxide to prepare a mixed liquid. The mixed liquid was stirred at room temperature for 24 hours. The resulting sodium chloride was removed by filtration, and the filtrate was concentrated under reduced pressure. Chloroform (100 ml) and a 10% by weight aqueous sodium hydroxide solution (100 ml) were added to the residue. After the organic layer was removed, concentrated hydrochloric acid was added to the aqueous layer in an ice bath until the aqueous layer became acid. The aqueous layer was extracted with chloroform, and then the solvent was removed. Subsequently, column purification was performed to thereby obtain 5.04 g of a triketone. This triketone was used in the next reaction without further purification. The triketone 5.04 g (30 mmol) and an acetic acid (50 ml) solution of aniline 2.81 ml (30.9 mmol) were stirred under heating at 80° C. for 3 hours. The reaction solution was neutralized with an aqueous saturated sodium hydrogencarbonate solution, and then the reaction product was extracted with chloroform. Chloroform was removed by concentration under reduced pressure, and the residue was subjected to column purification (developing solvent: heptane/ethyl acetate=4/1) to give 4.05 g (60%) of an intermediate [2]. The structure of the intermediate [2] was confirmed by NMR measurement.

¹H-NMR (400 MHz, CDCl3) δ: 2.05 (3H, s), 2.06-2.11 (2H, m), 2.46-2.54 (4H, m), 6.38 (1H, s), 7.21-7.26 (2H, m), 7.47-7.50 (3H, m).

### Synthesis of Intermediate [3]

Under nitrogen stream, a THF solution (2M, 5.7 ml, 11.3 mmol) of lithium diisopropylamide (LDA) was added dropwise to a THF solution (20 ml) including 1.70 g (7.55 mmol) of the intermediate [2] cooled to the temperature of −78° C. After stirring for one hour at the temperature of −78° C., 1.91 ml (22.65 mmol) of ally bromide was added thereto. The reaction solution was warmed to room temperature while stirring, and an aqueous ammonium chloride solution was added thereto, followed by extraction with chloroform. The organic layer was concentrated under reduced pressure, and the residue was subjected to column purification (developing solvent: heptane/ethyl acetate=8/1) to give 1.07 g (69%) of an intermediate [3]. The structure of the intermediate [3] was confirmed by NMR measurement.

¹H-NMR (400 MHz, CDCl3) δ: 1.83-1.90 (1H, m), 2.04 (3H, s), 2.11-2.27 (2H, m), 2.40-2.47 (1H, m), 2.52-2.55 (2H, m), 2.71-2.75 (1H, m), 5.01-5.08 (2H, m), 5.79-5.86 (1H, m), 6.37 (1H, s), 7.21-7.26 (2H, m), 7.43-7.52 (3H, m).

### Synthesis of Intermediate [4]

Palladium chloride (1.04 g, 5.88 mmol) was suspended in a mixed liquid of dimethylformamide (DMF, 40 ml) and distilled water (4 ml), and stirring was performed at room temperature for 5 minutes. A DMF solution (8 ml) including 1.51 g (5.88 mmol) of the intermediate [3] was added to the mixed liquid, and stirring was performed at room temperature for 15 hours. The reaction solution was subjected to Celite filtration, and water was added to the filtrate, followed by extraction with chloroform. The organic layer was concentrated under reduced pressure, and the residue subjected to column purification (developing solvent: heptane/ethyl acetate=3/1) to give 1.05 g (64%) of an intermediate [4]. The structure of the intermediate [4] was confirmed by NMR measurement.

¹H-NMR (400 MHz, CDCl3) δ: 1.79-1.90 (1H, m), 2.04 (3H, s), 2.11-2.17 (1H, m), 2.25 (3H, s), 2.32-2.38 (1H, m), 2.43-2.48 (1H, m), 2.69-2.72 (1H, m), 3.00-3.07 (1H, m), 3.20-3.25 (1H, m), 6.35 (1H, s), 7.21-7.23 (2H, m), 7.47-7.52 (3H, m).

### Synthesis of Intermediate [6]

A mixed liquid of 5.0 g (20 mmol) of 3-iodonitrobenzene (manufactured by Tokyo Chemical Industry Co., Ltd.), 12.8 g (200 mmol) of copper powder, and dimethylformamide (DMF, 50 ml) was stirred under heating at 200° C. for 10 hours. After cooling to room temperature, copper powder was removed by filtration, and water was added to the filtrate, followed by extraction with toluene. The organic layer was concentrated, and the residue was purified by recrystallization (heptane/toluene=10/1) to give 1.19 g (51%) of an intermediate [6]. The structure of the intermediate [6] was confirmed by NMR measurement.

¹H-NMR (400 MHz, CDCl3) δ: 7.70 (2H, t, J=8.0 Hz), 7.97 (1H, d, J=8.0 Hz), 8.30 (1H, d, J=8.0 Hz), 8.50 (1H, s).

### Synthesis of Intermediate [7]

Ethanol (20 ml), water (10 ml), and acetic acid (0.75 ml) were added to 1.0 g (4.1 mmol) of the intermediate [6], 318 mg (3.5 mmol) of calcium chloride, and 1.6 g (24 mmol) of zinc powder, and stirring was performed under heating at 80° C. for 3 hours. After the solid was removed by filtration, concentration was performed under reduced pressure. An aqueous sodium hydrogencarbonate solution was added to the residue, followed by extraction with ethyl acetate. The organic layer was concentrated under reduced pressure, and the residue was subjected to column purification (developing solvent: heptane/ethyl acetate=1/1) to give 650 mg (86%) of an intermediate [7]. The structure of the intermediate [7] was confirmed by NMR measurement.

¹H-NMR (400 MHz, CDCl3) δ: 3.71 (4H, br.s), 6.65 (2H, d, J=8.0 Hz), 6.87 (1H, s), 6.95 (1H, d, J=8.0 Hz), 7.19 (1H, t, J=8.0 Hz).

### Synthesis of Exemplary Compound (5)

In a 50-ml flask, 1.02 g (3.63 mmol) of the resulting intermediate [4], 320 mg (1.74 mmol) of the resulting intermediate [7], and 10 ml of acetic acid were placed, and stirring was performed under heating at 80° C. for 3 hours. After the reaction was completed, chloroform was added thereto, acetic acid was neutralized with an aqueous saturated sodium hydrogencarbonate solution, and the solvent was removed. Then, column purification (developing solvent: heptane/ethyl acetate=10/1) was performed, and recrystallization was performed with methanol to give 0.23 g of exemplary compound (5) as a white solid. The yield as 20%. The product was purified by sublimation (10⁻⁴ Pa, 300° C.). The structure of exemplary compound (5) was confirmed by NMR measurement.

¹H-NMR (400 MHz, DMSO-d6) δ: 1.93 (3H, s), 2.06 (3H, s), 2.29 (6H, s), 5.48 (2H, d), 6.40 (2H, s), 6.71 (2H, t), 7.15 (2H, dd), 7.30-7.38 (4H, m), 7.50-7.55 (8H, m), 7.76 (2H, m), 7.90 (2H, d), 8.02 (2H, d).

MALDI-TOFMASS (matrix-assisted laser desorption/ionization-time of flight mass spectrometry); 670 (M+)

### Example 2

The phosphorescence 0-0 band (T₁ energy level) at 77 K in a toluene solution (concentration: 10⁻³ mol/l) of the exemplary compound (5) obtained by the synthesis was measured with a fluorescence spectrophotometer (manufactured by Hitachi, Ltd., trade name: F-4500). As a result, the T₁ energy level was 417 nm.

### Example 3

Film formation was performed by a spin coating method, using a chloroform solution containing, at a concentration of 1% by weight, the exemplary compound (5) obtained by the synthesis. The HOMO energy level of the resulting film was measured with a photoelectron spectrometer in air (trade name: AC-2, manufactured by Riken Keiki Co., Ltd.), and the result was −5.42 eV.

### Example 4

### Fabrication of Organic Light-Emitting Device

An indium tin oxide (ITO) film was formed as an anode by sputtering with a thickness of 120 nm on a glass substrate. The resulting ITO film was patterned such that the electrode area was 4 mm². The substrate was subjected to ultrasonic cleaning using ultrapure water and isopropyl alcohol (IPA) in that order. Then, UV/ozone cleaning was performed, and the treated substrate was used as a transparent conductive supporting substrate.

A chloroform solution containing 0.3% by weight of N,N′-bis(9,9-dimethyl-9H-fluoren-2-yl)-N,N′-diphenyl-[1,1′-biphenyl]-4,4′-diamine represented by structural formula [Chem. 8] below was prepared and deposited by a spin coating method on the supporting substrate to form a hole injection/transport layer. The thickness of the hole injection/transport layer was set at 30 nm.

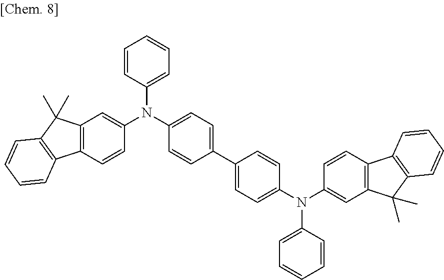 [SMILES: CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21]

Next, the exemplary compound (5) synthesized in Example 1, as a host material, and the phosphorescent Ir metal complex represented by structural formula [Chem. 9] below (synthesized according to the method described in Patent Literature WO2008/156879), as a guest material, were co-vapor-deposited on the hole injection/transport layer. In the co-vapor deposition, the vapor deposition rate was adjusted so that the concentration of the metal complex shown in [Chem. 9] was 15% by weight relative to the exemplary compound (5), and thereby a light-emitting layer with a thickness of 15 nm was provided. In the vapor deposition process, the degree of vacuum was 2.0×10⁻⁵ Pa, and the deposition rate was 0.2 nm/sec.

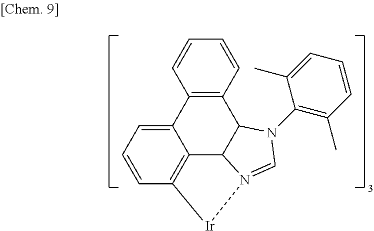

Furthermore, the pyridine compound (manufactured by Lumtec Corp.) represented by structural formula [Chem. 10] below was vapor-deposited on the light-emitting layer to form an electron transport layer with a thickness of 65 nm. In the vapor deposition process, the degree of vacuum was 2.0×10⁻⁵ Pa, and the deposition rate was 0.1 nm/sec.

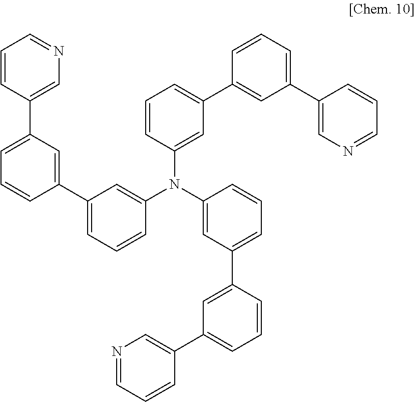 [SMILES: c1cncc(-c2cccc(-c3cccc(N(c4cccc(-c5cccc(-c6cccnc6)c5)c4)c4cccc(-c5cccc(-c6cccnc6)c5)c4)c3)c2)c1]

Next, lithium fluoride (LiF) was vapor-deposited with a thickness of 0.5 mm, and aluminum (Al) was further vapor-deposited with a thickness of 120 mm. The LiF/Al layer functions as a cathode opposite to the ITO anode. Thus, an organic light-emitting device was fabricated. In the vapor deposition process, the degree of vacuum was 4.0×10⁻⁵ Pa, and the deposition rate was 0.015 nm/sec for lithium fluoride and 0.4 to 0.5 nm/sec for aluminum.

The resulting organic light-emitting device was covered with a protective glass plate in a dry air atmosphere and sealed with an epoxy resin-based adhesive so as to prevent degradation of the device due to adsorption of moisture.

### Evaluation of Device

When the device thus obtained had a luminance of 500 cd/m², in which the ITO electrode (anode) was set as a positive electrode, and the LiF/Al electrode (cathode) was set as a negative electrode, the applied voltage was measured to be 4.0 V. The luminous efficiency was 13.51 m/W, and blue emission was observed.

### Comparative Example 1

For comparison, comparative compound (1) (trade name: 4,4′-N,N′-dicarbazolyl-m-biphenyl (synonym: mCBP)), i.e., a known typical carbazole compound, was used. The structural formula thereof is shown below. A device was fabricated as in Example 1 except that comparative compound (1) was used instead of exemplary compound (5), and evaluation was performed in the same manner. When the device had a luminance of 500 cd/m², the applied voltage was measured to be 4.0 V. The luminous efficiency was 11.51 m/W, and blue emission was observed.

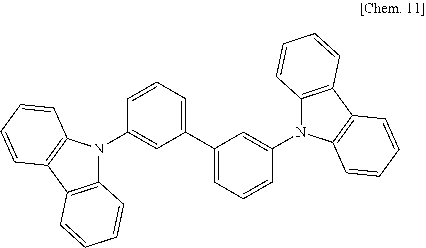 [SMILES: c1cc(-c2cccc(-n3c4ccccc4c4ccccc43)c2)cc(-n2c3ccccc3c3ccccc32)c1]

### Comparative Compound (1)

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-153989, filed Jul. 6, 2010, which is hereby incorporated by reference herein in its entirety.

### INDUSTRIAL APPLICABILITY

The technique of the present invention can be used not only for display apparatuses such as full-color displays, but also for illumination apparatuses, apparatuses using photoelectric conversion elements, electrophotographic apparatuses, and the like.

1. A pyrroloindole compound represented by general formula (1): 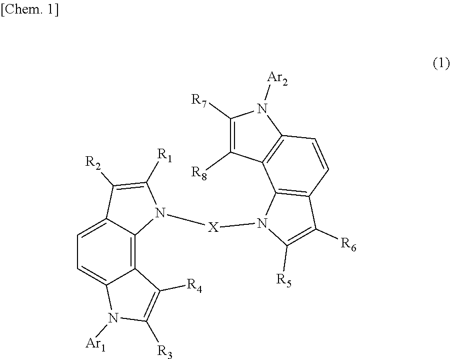
wherein, in general formula (1), X represents a substituted or unsubstituted arylene group, Ar₁ and Ar₂ each represent a substituted or unsubstituted aryl group, and R₁ to R₈ each represent a hydrogen atom or an alkyl group having 1 to 2 carbon atoms.

2. An organic light-emitting device comprising at least one organic layer disposed between a pair of opposing electrodes, wherein at least one of the at least one organic layer is a light-emitting layer containing the compound according to claim 1.

3. The organic light-emitting device according to claim 2, wherein the light-emitting layer contains, as a guest material, a phosphorescent Ir metal complex, and contains, as a host material, the pyrroloindole compound represented by general formula (1).

4. An image display apparatus comprising:
the organic light-emitting device according to claim 2; and
a thin-film transistor configured to apply an electrical current to the organic light-emitting device.
